(19) United States
(12) Patent Application Publication
(10) Pub. No.: US 2024/0180559 A1
(43) Pub. Date: Jun. 6, 2024

(54) LEFT ATRIAL AURICLE OCCLUDER

(71) Applicant: Riccardo LAURENTI, Borgomasino (Torino) (IT)

(72) Inventors: Riccardo LAURENTI, Borgomasino (Torino) (IT); Valerio VALLANA, Torino (IT)

(21) Appl. No.: 18/553,320

(22) PCT Filed: Mar. 22, 2022
(86) PCT No.: PCT/IB2022/052584

§ 371 Date: Sep. 29, 2023

(30) Foreign Application Priority Data
Apr. 2, 2021 (IT) 102021000008411

(51) Int. Cl. A61B 17/12; A61B 17/00

(52) U.S. Cl. CPC A61B 17/12122; A61B 17/12172; A61B 2017/00867

(57) ABSTRACT

Left atrial auricle occluder designed to be implanted into a left atrium through a transcatheter route includes a self-expandable hyperelastic cage configured to rest against an inner surface of the left atrium and is provided with an end obturator suitable to be retained frontally against a rim of an auricle, plugging it, by an elastic thrust exerted by the cage.

### FIELD OF THE INVENTION

The present invention generally relates to cardiac prostheses and more particularly it relates to a left atrial auricle occluder.

Auricles (right and left, but the left auricle in this case which is far more important) are protuberances of the atrium which are vastly differently shaped depending on individuals, but connected to the atrium by a vaguely circular opening, of variable shape and size.

Given that it is only an ancestral remnant of evolution, the auricle does not have a specific function. On the contrary, given that it is basically a “sac” without outlet in which the blood tends to stagnate, it can be a site of clots especially if the from atrial fibrillation, a patient is suffering pathological condition that facilitates the formation of thrombi (clots that detach and enter into the circulatory system). As a matter fact in patients with atrial fibrillation, more than 90% of atrial thrombi originate from the left auricle. Therefore, occlusion of the left auricle is indicated in patients with permanent atrial fibrillation, high risk of stroke and contraindications to anticoagulants.

### STATE OF THE ART

The remedies are related to the diseases of the patient and there are two treatment options:
pharmacological: while on the one hand it makes blood clotting more difficult, it makes the possibility of bleeding more frequent;
mechanical: provides for the use of filters or plugs which filter or obstruct clot generation sites.

As mentioned, given that the auricle has no function, the best mechanical solution is the definitive closure of the connection hole with a kind of stable “plug” that ensures the complete isolation of the atrium pouch. If the blood exchange between the cavity and the atrium is completely excluded, the auricle will be filled with coagulated blood, which will gradually turn into tissue without any consequence. This solution is the only viable solution when the pharmacological solution is not advisable due to concomitant diseases which result in a high risk of bleeding. Furthermore, it should be observed that in some cases the pharmacological therapy is in itself ineffective due to insufficient reaction to the active ingredient by the patient.

Though apparently being a simple problem, the mechanical closure that ensures the hydraulic sealing in the specific anatomical conditions of the auricle (non-coplanarity of the inlet surface, irregularity of the shape of the orifice, unpredictability of the configuration), as well as the stability of the fixation of the prosthesis, it is a problem that is difficult to solve and is still not fully solved.

Current solutions provide for prostheses that are based on a three-dimensional shape of the occluder that typically includes a sealing part and an anchoring part and it is implanted by inserting it through the transcatheter route into the left atrium, entering into the auricle to close it from the inside. These prostheses are then held in position with the essential aid of hooks or, alternatively, of a self-expandable circular element made of metal. Solutions of this type are described and illustrated, for example, in documents EP2716237, EP3146915 and EP3329865, EP3369388 and EP3398536.

These solutions are constructively complex and reveal reliability problems in relation to the hermetic sealing of the auricle. Furthermore, the intervention for the implantation thereof could be complicated.

A solution in which the occluder includes an elastic cage is described and illustrated in US2015/22382. This solution is not capable of ensuring the required stability of the occluder following the application thereof.

### SUMMARY OF THE INVENTION

The object of the present invention is to overcome the drawbacks of the known solutions and to provide a simple left atrial auricle occluder that is functional and reliable, in particular in relation to the stability thereof in the applied condition.

The invention is based on a peculiar concept completely different from the prior art, given that it provides for the watertight sealing of the auricle from the inside of the atrium, closing it frontally, rather than from the inside.

To this end, the occluder according to the invention comprises a self-expandable hyperelastic cage prosthesis configured to occupy at least a substantial part of the atrium resting against the inner surface of the atrium and provided with an end obturator suitable to be retained, i.e. urged, frontally against the auricle, plugging it, by the elastic thrust exerted by said cage.

The obturator, typically consisting of a patch, may have a generally conical or ogival shape and it advantageously has dimensions such to frontally cover the entire connection hole between the atrium and the auricle with redundancy.

Following the expansion thereof, the cage conveniently has a generally ellipsoidal shape and it typically consists of an annular network of longitudinal wires having a wavy or sinusoidal arrangement. In this case the crests of the wavy or sinusoidal wires are juxtaposed and can be free or mutually joined.

In one embodiment, the cage is configured to occupy substantially the entire volume of the atrium, while in a variant the cage is configured to occupy only part of the volume of the atrium.

Furthermore, the cage is conveniently provided with a passage to allow, once implanted, the possible access to the mitral valve in case of need.

### BRIEF DESCRIPTION OF THE DRAWINGS

The invention will now be described in detail with reference to the attached drawings, provided purely by way of non-limiting example, wherein:

FIG. 1 is a cross-sectional schematic view of a heart within which a left atrial auricle occluder according to the invention is implanted,

FIG. 2 is a perspective view and in larger scale of the occluder,

FIG. 3 is a side elevational view of the occluder,

FIG. 4 is a front elevational view and in enlarged scale of the occluder,

FIG. 5 is a perspective view of a variant of the occluder according to the invention,

FIG. 6 is a front elevational view of the occluder of FIG. 5, and

FIG. 7 is a side elevational view of the occluder of FIG. 5.

### DETAILED DESCRIPTION OF THE INVENTION

In summary, the atrial auricle occluder according to the invention provides for a flat-shaped, but highly flexible and adaptable disc, typically made of tissue, that is larger than the opening of the auricle to be closed and such to cover it all with sufficient redundancy. The disc takes a conical or ogival shape and it is stopped against the opening by a cage element made of superelastic metal which rests on the inner surface of the left atrium and it is introduced through the percutaneous route.

The optimal access is thought to be the usual one through the venous system, passing through the inter-atrial septum by means of an instrument, not subject of the invention, which allows direct access to the left atrium from a position directly opposite to that of the auricle opening.

An important additional element may be the presence of a hole with appropriate size located on the side of the cage (which, following the shape of the atrium, will have an ellipsoidal configuration) in a position corresponding to the mitral valve, in anticipation of a replacement or repair of the valve.

The ellipsoid could be open at the end corresponding to the transseptal access to allow further subsequent accesses to the site or, possibly, for a better repositioning of the system.

It is also appropriate to provide for that the ellipsoid has, at the support on the occluder, a protuberance in the form of a cone of modest depth which serves as a guide element and aid for correct positioning.

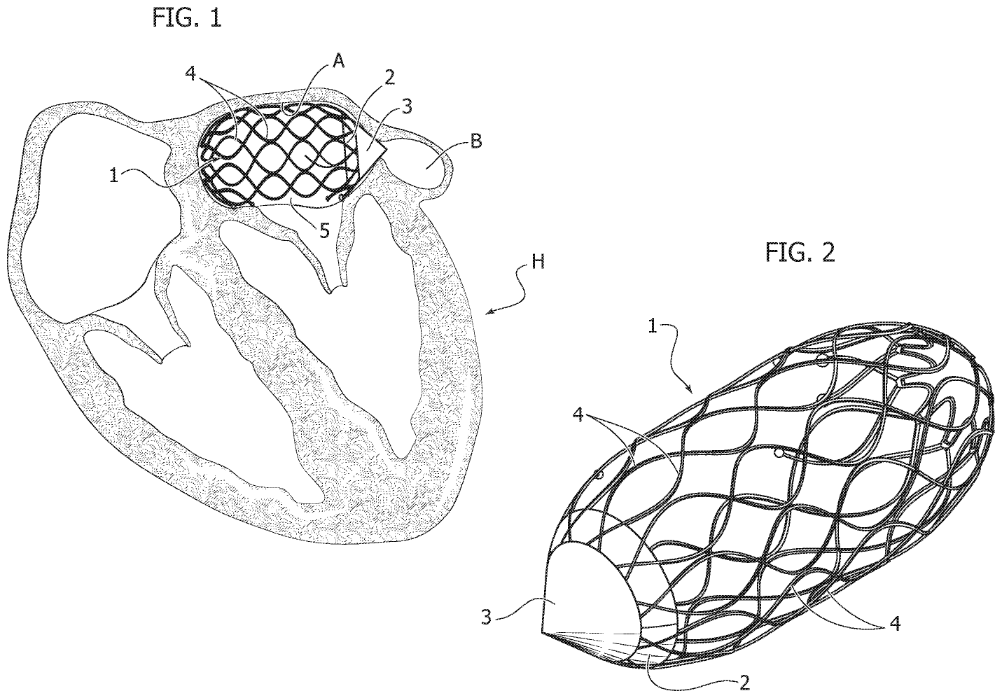

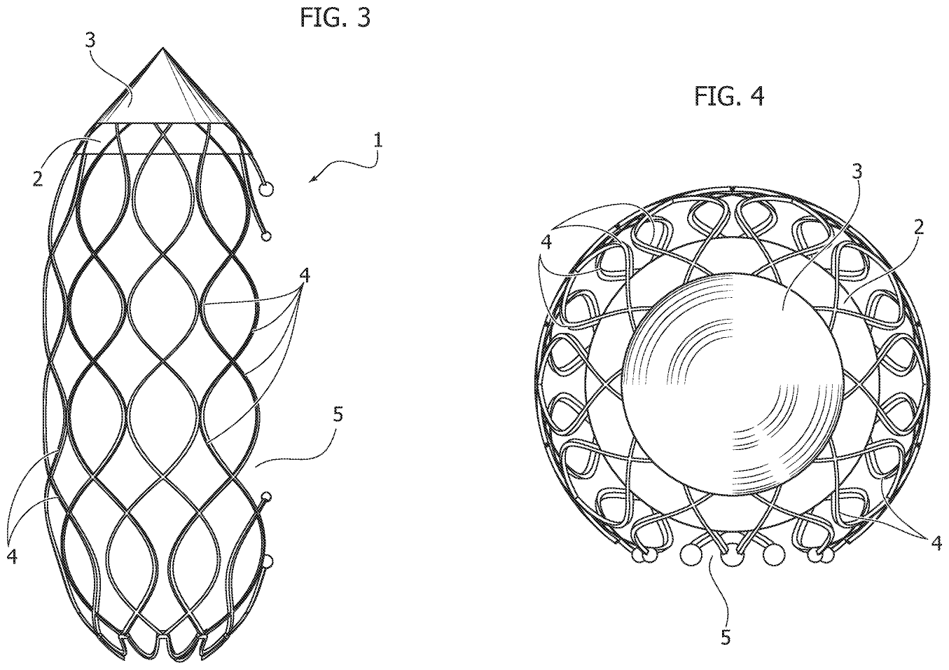

Referring now in detail to FIGS. 1 to 4, the occluder according to the invention is shown in the implanted and deployed configuration thereof inside the left atrium A of the heart H. The occluder consists of a self-expandable hyperelastic cage 1 configured to rest against the inner surface of the atrium A and provided at the front end 2 thereof with an obturator 3 suitable to be retained or urged frontally against and within the rim of the auricle B, plugging it, by the elastic thrust exerted by the cage 1.

In the case of the example described now, the front end 2 of the cage 1 has a conical shape and the obturator 3 consists of a flexible patch, typically made of tissue as mentioned, and adaptable thanks to the elasticity of the cage 1 so as to seal the opening of the auricle B. To this end, the size of the obturator 3 are such to cover the rim of the opening of the auricle B with redundancy, that is beyond its perimeter.

The cage 1 has a generally ellipsoidal shape and it occupies at least a substantial part of the left atrium A: in this example it is sized to occupy substantially the entire volume of the left atrium A, resting and reacting stably against the inner wall thereof so as to elastically press the obturator 3 against the auricle B, closing it substantially hermetically.

The cage 1 consists of an annular network of longitudinal wires 4 having a wavy or sinusoidal arrangement. The crests of the wavy or sinusoidal wires are juxtaposed and can be free or mutually joined. Typically, the superelastic material of the wires 4 may be an SMA of nickel and titanium (Nitinol).

The network forming the cage 1 is conveniently provided with a lateral passage 5 to allow, in the implanted condition of the occluder 1, the possible access to the mitral valve.

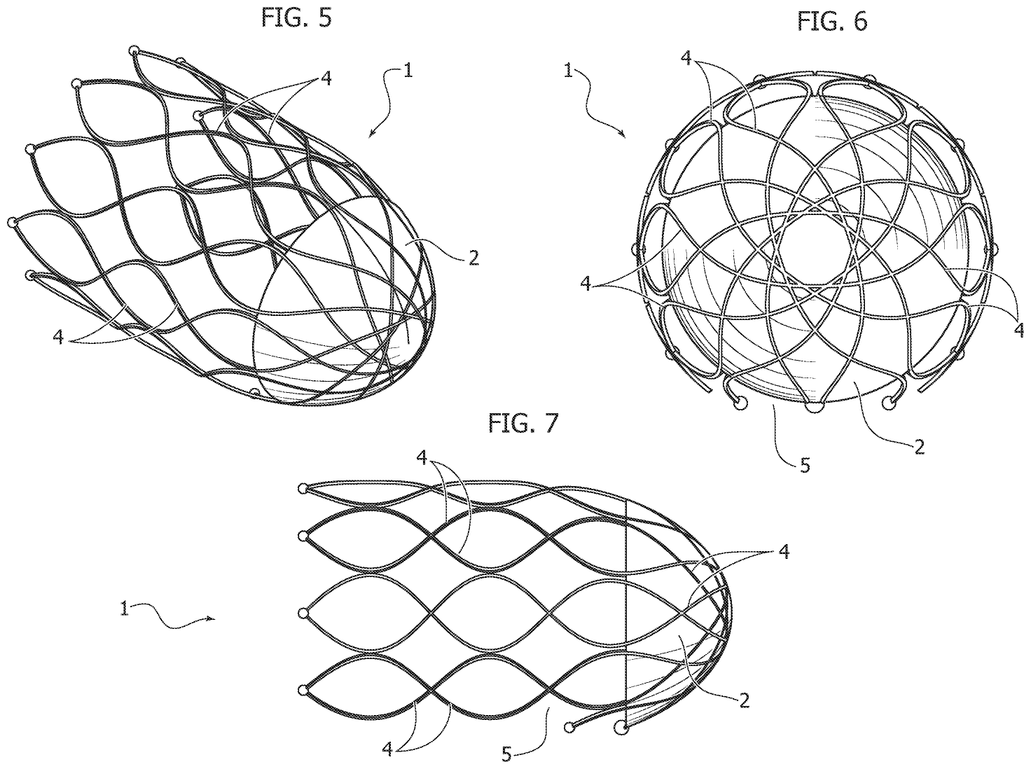

The variant shown in FIGS. 5-7 is generally similar to the previous embodiment, with the difference relating to the size of the cage 1 which in this case is configured so as to occupy only part (for example slightly more than half) of the volume of the atrium A. Furthermore, the front end 2 of the cage 1 onto which the patch 3 is applied (arranged inside the cage 1 instead of outside as in the previously described embodiment) has an ogival, or rounded shape, rather than a conical shape.

In this variant, the dorsal end of the cage 1 is open to allow further subsequent accesses to the site or, possibly, for a better repositioning of the occluder.

In further variants not shown, the cage may have geometrical shapes other than the ellipsoid, provided that they are suitable to achieve the same functional sealing effect of the obturator 3 against the rim of the auricle B. For example, the cage may have an umbrella-shaped configuration.

Obviously, the construction details and the embodiments may widely vary with respect to what has been described and illustrated, without departing from the scope of protection of the present invention as defined in the claims that follow.

1. A left atrial auricle occluder configured to be implanted into a left atrium through a transcatheter route, the occluder comprising;
a self-expandable hyperelastic cage configured to occupy at least a substantial part of the left atrium resting against an inner surface of the left atrium and provided with an end obturator configured to be retained frontally against a rim of an auricle, plugging it, by an elastic thrust exerted by said cage.

2. The occluder according to claim 1, wherein the obturator has a generally conical or ogival shape and has a size configured to cover the rim of the auricle with redundancy.

3. The occluder according to claim 1, wherein the cage, following an expansion thereof, has a generally ellipsoidal shape.

4. The occluder according to claim 1, wherein the cage is formed by an annular network of longitudinal wires having a wavy or sinusoidal arrangement.

5. The occluder according to claim 4, wherein crests of the wavy or sinusoidal wires are juxtaposed and they are free or mutually joined.

6. The occluder according to claim 1, wherein the cage is sized to occupy substantially an entire volume of the left atrium.

7. The occluder according to claim 1, wherein the cage is sized to occupy only part of a volume of the left atrium.

8. The occluder according to claim 1, wherein the cage is open at an end opposite to said obturator.

9. The occluder according to claim 1, wherein the cage is provided with a lateral passage to allow, in an implanted condition of the occluder, access to a mitral valve.
